(19) United States
(12) Patent Application Publication
(10) Pub. No.: US 2026/0248991 A1
(43) Pub. Date: Aug. 27, 2026

(54) HERNIA DETECTING BELT AND PERITONEAL DIALYSIS SYSTEM INCORPORATING SAME

(71) Applicants: Fresenius Medical Care Holdings, Inc., Waltham, MA (US); Fresenius Medical Care Deutschland GMBH, Bad Homburg (DE)

(72) Inventors: David Yuds, Hudson, NH (US); Martin Crnkovich, Walnut Creek, CA (US); Christian Schlaeper, Wehrheim (DE)

(73) Assignees: Fresenius Medical Care Holdings, Inc., Waltham, MA (US); Fresenius Medical Care Deutschland GMBH, Bad Homburg (DE)

(21) Appl. No.: 19/061,473

(22) Filed: Feb. 24, 2025

(51) Int. Cl. A61M 1/28; A41D 13/005; A41D 13/12

(52) U.S. Cl. CPC A61M 1/28; A41D 13/0051; A41D 13/1281; A61M 2202/0021; A61M 2202/04; A61M 2205/0216; A61M 2205/3327; A61M 2205/3375; A61M 2205/36; A61M 2209/088; A61M 2210/10; A61M 2230/005

(57) ABSTRACT

A sensor belt for peritoneal dialysis is provided, as are methods for peritoneal dialysis. The sensor belt can be wrapped around and worn by a patient during a peritoneal dialysis treatment. The belt includes a sensor panel having an ultrasonic sensor secured to, or in, a middle flap. The ultrasonic sensor is configured to be aligned with and surround a peritoneal dialysis access and/or surrounding abdominal region when the patient wears the belt. Ultrasonic signals sent by a transmitter of the ultrasonic sensor, reflected from the patient's abdominal musculature, and received by a receiver of the ultrasonic sensor, are used to detect hernias or symptoms of an oncoming hernia, particularly in the vicinity of the peritoneal dialysis access.

### FIELD OF THE INVENTION

The present invention relates to peritoneal dialysis and hernia detection.

### BACKGROUND OF THE INVENTION

A hernia is an uncomfortable and potentially dangerous bulge in the intestinal wall where the lining of the abdominal cavity (peritoneum) protrudes between weak muscles or through a hole in the abdomen. People who have hernias typically cannot play tennis or go bowling. It is also difficult for some hernia patients to stand up, bend over, run, or even crawl. Hernia patients that also undergo peritoneal dialysis (PD) face even greater hurdles for and during therapy. Sometimes, a hernia can be the ruination of the PD therapy, and a hernia operation is needed before PD therapy can be resumed.

Hernias are a common problem for PD patients. Currently, the only way to reduce the risk of causing a hernia is to use largely inefficient low volume, recumbent only (LVRO) PD therapies. Even with LVRO therapies, however, there is no way to monitor patients to ensure the pressures from fill rates and volumes are appropriate or whether the intestinal wall is straining.

Hernias are commonly cited by PD patients listing their reasons for dropping out of PD therapy. Furthermore, new approaches to home dialysis like Urgent Start PD must rely on low fill volumes of PD solution with short dwell times. These conditions are needed to ease an end stage renal disease (ESRD) patient, into home dialysis, but require significant labor from specially trained nurses during a ramp-up stage. Similarly, LVRO PD is prescribed for PD patients who are getting back into PD after a surgery to repair a hernia. Not only is LVRO inefficient, LVRO may not be able to adequately support patients with low glomerular filtration rates. This means that ESRD patients are more likely to be sent to a dialysis center for hemodialysis therapy, making it more difficult and more costly to later transition to home therapies like PD.

Other problems associated with hernias in PD patients include leakage within 30 days of PD catheter insertion. Leakage is when fluid from inside the peritoneum leaks out through an opening in the body. Additionally, there is a risk of leakage from the catheter exit site after trauma and peritonitis. Furthermore, PD patients who have recently had surgery are often on stringent lifting restrictions that can be as low as five pounds. A 6-liter (6 L) bag of PD solution, however, weighs thirteen (13) pounds. As such, PD patients are often subjected to risks just by performing their PD therapy.

There is a need and desire to provide PD patients with hernia detecting capabilities, particularly during a PD therapy session.

There is also a need and desire to closely monitor a patient's peritoneum during a PD therapy session so that a hernia can be detected or avoided.

### SUMMARY OF THE INVENTION

It is an object of the present invention to provide a sensor belt for use by a patient during peritoneal dialysis, which incorporates an ultrasonic sensor to closely monitor the patient's peritoneum, detect a hernia, and detect anomalies in the patient's musculature that indicate a propensity for a hernia to form.

It is an object of the present invention to improve monitoring of the PD catheter exit/injection site, herein called a PD access, of a PD patient. The monitoring is accomplished by using a sensor belt of the present invention, which incorporates one or more ultrasonic sensors into a belt proper.

It is an object of the present invention to provide a sensor belt that comprises an array of ultrasonic sensors, which array can be configured to fit around the PD access of a PD patient. In addition to providing structural support for the patient's abdomen, the one or more ultrasonic sensors can monitor muscle tissue adjacent to the PD access for signs of strain, a hernia, early indications of rupture, and the like.

It is an object of the present invention to provide an array of ultrasonic sensors inside a sensor belt, which can be moved to align with a PD access of a patient wearing the sensor belt. Additional ultrasonic sensors can be placed in other strategic locations.

It is an object of the present invention to provide a sensor belt that comprises a PD catheter retainer configured to hold the end of a PD catheter tube securely in place in a pouch of the sensor belt.

It is an object of the present invention to provide a sensor belt for PD, which comprises a wetness sensor integrated into the sensor belt to monitor the PD access for leakage.

The foregoing and further objects of the present invention are provided by a sensor belt for use by a peritoneal dialysis (PD) patient, during a peritoneal dialysis therapy on the patient. The sensor belt comprises a plurality of closure panels and a sensor panel that fasten to one another to secure the sensor belt around the back and abdomen of the patient. The sensor belt is configured to be fastened around the back and abdomen of the patient without pressing on the PD patient's PD access. The sensor panel can then be secured to cover the PD access and align one or more ultrasonic sensors with the PD access and surrounding musculature of the patient's abdomen.

The sensor belt can comprise a belt proper comprising a mid-length section having a length, a first side panel having a length, a second side panel having a length, and the middle flap. The one or more ultrasonic sensors can be secured to the middle flap to form the sensor panel and the sensor belt can be configured such that, during use, the ultrasonic sensor is aligned with, for example, surrounds, the peritoneal dialysis access of the patient when the sensor belt is worn by the patient during PD therapy.

The foregoing and other objects of the present invention will be explained in greater detail below and with reference to the accompanying drawings.

### BRIEF DESCRIPTION OF THE DRAWINGS

The invention may be more fully understood with reference to the accompanying drawings. The drawings are intended to illustrate, not limit, the present teachings.

FIG. 1A is a front view of a sensor belt in a laid-flat configuration, according to an embodiment of the present invention.

FIG. 1B is an enlarged sectional view, in partial breakaway, of section 1B shown in FIG. 1A.

FIG. 2 depicts a first step of a method according to an embodiment of the present invention, of fitting the sensor belt shown in FIGS. 1A and 1B, around the back and abdomen of a peritoneal dialysis patient.

FIG. 3A depicts an intermediate step of the method, subsequent to the first step shown in FIG. 2, wherein upper closure panels of a first side panel and of a second side panel have been wrapped around the patient and fastened to each other, and lower closure panels of the first side panel and of the second side panel have been wrapped around the patient and fastened to each other.

FIG. 3B depicts an intermediate step of the method, subsequent to the step shown in FIG. 3A, wherein a catheter retainer has been fastened to the overlapping and fastened upper closure panels of the first side panel and of the second side panel.

FIG. 4 is an illustration of the sensor belt shown in FIGS. 1A, 1B, 2, 3A, and 3B fastened around the back and abdomen of the patient with a sensor panel and a middle flap of the second side panel being fastened to one another such that the ultrasonic sensor is aligned with the PD access and surrounding abdominal region, of the patient.

FIG. 5 is a close-up, broken away view, in partial cutaway, of a sensor panel according to an embodiment of the present invention.

### DETAILED DESCRIPTION OF THE INVENTION

The present invention provides a sensor belt for use by a peritoneal dialysis (PD) patient, during a peritoneal dialysis therapy on the patient. The sensor belt can be a support belt. The sensor belt is configured to be worn by the patient, around the patient's back and abdomen. The sensor belt can comprise a plurality of flaps or panels that fasten to one another to secure the sensor belt around the back and abdomen of the patient. The PD patient has a peritoneal dialysis access formed through a region of the patient's abdomen and the sensor belt can be configured to be fastened around the back and abdomen of the patient without covering the access.

The sensor belt can comprise a belt proper comprising a mid-length section having a length, a first side panel having a length, a second side panel having a length, and a middle flap. The belt proper has an overall length that is defined by the sum of the lengths of the mid-length section, the first side panel, and the second side panel. The sensor belt also comprises an ultrasonic sensor secured to the middle flap of the belt proper such that the ultrasonic sensor secured to the middle flap comprises a sensor panel. The sensor belt is configured such that, during use, the ultrasonic sensor is aligned with, for example, surrounds, the peritoneal dialysis access and/or surrounding abdominal region of the patient, when the sensor belt is worn by the patient.

The mid-length section can be configured to contact the back of the patient while the patient is wearing the sensor belt. The first side panel and the second side panel can be provided with cooperating fasteners or fastener portions that are configured to secure the first side panel to the second side panel at the abdomen of the patient while the patient is wearing the sensor belt. The first side panel can comprise an upper closure panel and a lower closure panel. The upper closure panel can be configured to wrap at least partially around the abdomen of the patient, above the peritoneal dialysis access, and fasten to the second side panel, while the patient is wearing the belt. The lower closure panel can be configured to wrap at least partially around the abdomen of the patient, below the peritoneal dialysis access, and fasten to the second side panel, while the patient is wearing the belt. The upper closure panel and the lower closure panel can be spaced apart from each other at the peritoneal dialysis access, such that neither the upper closure panel nor the lower closure panel covers the peritoneal dialysis access while the patient is wearing the belt. The sensor panel, including the middle flap, can be configured to wrap at least partially around the abdomen of the patient, cover the peritoneal dialysis access, and align the ultrasonic sensor with the region of the abdomen where the peritoneal dialysis access is located, while the patient is wearing the belt. The sensor panel can be secured to a middle flap of the second side panel, to the upper or lower closure panels of the second side panel, to the upper or lower closure panel of the first side panel, or to a combination thereof.

Each of the first side panel and the second side panel can independently be made of a stretchable material. The fasteners, cooperating fasteners, or fastener portions can comprise at least one patch of hook fasteners and at least one patch of loop fasteners. The fasteners, cooperating fasteners, or fastener portions can comprise one or more of a hook and loop fastener, a button, a zipper, a snap fastener, a magnetic fastener, and a lace.

The ultrasonic sensor can comprise an array of ultrasonic sensors, for example, an array arranged in the formation of a circle. The array can surround the peritoneal dialysis access at the region of the abdomen when the patient is wearing the sensor belt.

The sensor belt can comprise one or more rigid vertical support ribs that are configured to provide back support to the patient. The mid-length section can comprise one or more vertical sleeve pockets containing, respectively, the one or more rigid vertical support ribs, such that the mid-length section is in the form of a ribbed support section. The one or more rigid vertical support ribs can be removable from the respective one or more vertical sleeve pockets.

The second side panel can comprise an upper closure panel and a lower closure panel. The upper closure panel of the second side panel can be configured to wrap at least partially around the abdomen of the patient, above the peritoneal dialysis access, and fasten to the first side panel, while the patient is wearing the belt. The lower closure panel of the second side panel can be configured to wrap at least partially around the abdomen of the patient, below the peritoneal dialysis access, and fasten to the first side panel, while the patient is wearing the belt. The sensor belt can be configured such that neither the upper closure panel of the second side panel nor the lower closure panel of the second side panel has a length sufficient to wrap far enough around the abdomen of the patient to reach the PD access, for example, so that only the closure panels of the first side panel extend to and past the PD access.

During use, the upper closure panel of the first side panel and the lower closure panel of the first side panel are vertically separated from one another by a gap, at the peritoneal dialysis access. The sensor panel can be configured to overlap the gap and align the ultrasonic sensor with the peritoneal dialysis access.

The first side panel can be connected to the mid-length section at a first vertical seam, and the second side panel can be connected to the mid-length section at a second vertical seam. The middle flap can be connected to the mid-length section at or coincident with the first vertical seam or the second vertical seam.

The ultrasonic sensor can comprise an array of ultrasonic sensors arranged in a circle. A through-hole can be provided through a middle of the circle for accommodating a catheter tube.

The combination of a sensor belt for peritoneal dialysis, as described herein, fastened around an abdomen, is also provided by the present invention, as is a method for fastening the sensor belt to a patient. A method for peritoneal dialysis treatment of the patient, is also provided. For fastening, the method can comprise placing the mid-length section against the back of the patient, wrapping the first side panel and the second side panel around the abdomen of the patient, and fastening together the first side panel and the second side panel around the abdomen of the patient. The method can then further involve wrapping the sensor panel at least partially around the abdomen of the patient and over the PD access such that the ultrasonic sensor is aligned with the abdominal region surrounding the PD access.

The method can also comprise sending ultrasonic waves from the ultrasonic sensor toward the region of the abdomen where the PD access is located. The method can comprise receiving, with the ultrasonic sensor, ultrasonic signals reflected from musculature within the abdomen of the patient. The method can further comprise carrying out a peritoneal dialysis treatment on the patient, through the PD access, while receiving, with the ultrasonic sensor, ultrasonic signals reflected from the musculature within the abdomen of the patient. The method can comprise modifying the peritoneal dialysis treatment, based on the ultrasonic signals received.

The sensor belt can comprise a heater, and the method can further comprise heating the sensor belt during a peritoneal dialysis treatment. The sensor belt can comprise a wetness detector, and the method can further comprise detecting wetness at or adjacent to the PD access during a peritoneal dialysis treatment.

A method for carrying out a peritoneal dialysis treatment on a patient, is also provided. The method comprises fastening a sensor belt as described herein, around the abdomen of a patient, aligning the ultrasonic sensor of the sensor belt with a peritoneal dialysis access formed in the abdomen of the patient, and carrying out a peritoneal dialysis treatment on the patient by transferring a peritoneal dialysis solution through the peritoneal dialysis access formed through the patient's abdominal wall. The method further comprises sending and receiving ultrasonic signals from and to the ultrasonic sensor, wherein the ultrasonic signals received pertain to the muscle tissue, i.e., musculature, of the abdomen of the patient, particularly near and around the PD access. Depending upon whether or not the ultrasonic signals show a hernia or stretching of the musculature to an extent that might lead to a hernia, the method may or may involve modifying the peritoneal dialysis treatment based on the ultrasonic signals received.

The incorporated ultrasonic sensors in the sensor belt fit around or otherwise surround the PD patient's PD access and monitor muscle tissue for signs of strain or the earliest indications of rupture. The belt fits around the patient's waist, wraps from the back to the front, and is fastened by fasteners that avoid the PD access so as not to occlude a PD catheter or tubing connected to or that is part of the PD access. The sensor belt comprises a PD catheter retainer that holds the end of the catheter securely in place in a pouch. The ultrasonic sensor or sensor array can be moved to align with the PD access of the patient and be focused on the muscle wall around the most vulnerable location, that is, the PD access and the closely adjacent abdominal region. Additional ultrasonic sensors can be placed in other strategic locations. The wetness sensor can be integrated into the sensor belt or ultrasonic sensor or sensor array to monitor the PD access for leakage.

In an exemplary therapy, the PD patient's physician can instruct the patient to wear the belt during the first 30 days, for example, of an urgent start PD therapy, or during the first 30 days following a physical trauma to the patient's abdomen, or an infection. A wireless link to electronics in the sensor belt enables the physician and the patient to monitor the PD access and surrounding abdominal region. The patient can input data about the experience. Pulse, temperature, and belt tension sensors can also be incorporated to monitor the patient during the PD therapy and can be used to further support the system in learning the strain put on the patient through various daily activities like working, shopping, and exercising.

A hernia detecting system is also provided that links to the patient's automated peritoneal dialysis (APD) cycler to exchange data on the fill flow rate and PD solution dwell time, to calibrate the patient's experience and use models and machine learning to determine recommendations for changes to therapy to make it more efficient while staying within set limits. In the event that the hernia detecting system senses leakage not related to perspiration, detects muscle strain or rupture, or detects a weakening of the muscle tissue, the system is configured to send out an alert to the patient, the patient's physician, and/or a care team. The alert can be configured to display on the APD cycler and trigger a partial drain, by the cycler, to immediately relieve pressure. An alert can also be used to make an adjustment to the fill volume.

A tilt and motion sensor can be incorporated into the sensor belt and used to monitor the patient's position while sleeping. The hernia detecting system can be configured to provide tips for an improved recovery by changing positions with subtle sounds or vibrations to roll over. If the linked APD cycler is detecting a slow fill or drain phase during a PD therapy, the APD cycler can trigger the belt to vibrate or beep to encourage the patient to change positions, which can be particularly helpful in cases where tubing has become kinked.

The hernia detecting system can also be configured to enable the patient's physician to remotely tighten the upper and lower sensor limits based on medical record inputs, for example, inputs such as those denoting that the patient has had an infection or experienced some trauma while not wearing the belt. Likewise, as the patient grows stronger (as measured by the belt), the physician can loosen the limits to allow for more physical activity and larger fill volumes, contributing to a more efficient PD therapy, and sooner than is possible with conventional monitoring.

The sensor belt can also integrate heat therapy to facilitate faster healing. A user-settable temperature and duration can be utilized to ensure patient comfort. The hernia detecting system can monitor the progression of the recovery based on the addition of the applied heat.

The sensor belt can comprise one or more panels or layers made of or comprising a stretchy, comfortable material that conforms to the user's abdomen and back. A pouch can be formed in the front of the sensor belt between the inner layer and outer layer can have a low-profile pouch fastener at or adjacent a top of the pouch. One or more plastic snap fasteners can be used, or hook and loop fasteners can be used, to secure the sensor belt around the back and abdomen of the user.

One or more sections or panels of the sensor belt can comprise two or more layers. An inner layer of material and an outer layer of material can be connected together. The connection of the two layers can also provide a low-profile, for example, a line of stitching can be used. The outer layer of material, in particular, can comprise a stretchable material. The length, height, and relative dimensions of the sensor belt can be customized to closely match the back, abdomen, and peritoneal dialysis access of a particular patient for whom the sensor belt is particularly designed.

The sensor belt has a bottom, a top, an overall length, and a height. The height of the belt can be four inches or more, for example, at least eight inches, at least ten inches, at least 12 inches, at least 15 inches, from eight inches to 25 inches, from ten inches to 20 inches, from 12 inches to 18 inches, or the like. Depending on the size of the patient for whom the sensor belt is intended, the overall length of the sensor belt can vary. For a very large patient, for example, having a waist size of greater than 40 inches, the overall length of the sensor belt can be at least 42 inches, at least 45 inches, at least 50 inches, at least 55 inches, or the like.

The belt can have a height of at least four inches along the front, where the belt touches the patient's abdomen. The height in the front can be, for example, a height of five inches, a height of six inches, a height of eight inches, a height of ten, or the like. The height of the remainder of the belt does not necessarily need to be the same as the height at the front of the belt. In the back, the sensor belt comprises a mid-section panel that can have a shorter height, the same height, or a greater height than the height at the front of the belt.

A through-hole can be formed through one or more closure panels or middle flaps, at the front of the sensor belt, during use. The through-hole can be sized to accommodate a PD catheter. The through-hole can have a dimeter that is large enough or stretchable enough to enable a catheter to be fed through the through-hole, the patient's PD access to outside of the belt. The through-hole can be at least 1/16 inch in diameter, at least ⅛ inch in diameter, at least ¼ inch in diameter, or the like. Instead of a through-hole, a slit or other opening can be provided in one or more of the closure panels or middle flaps, at the front of the sensor belt, during use. The through-hole or opening can be positioned, for example, at or below the midline of the belt.

The sensor belt can comprise one or more fasteners. Single fasteners or a set of fastener components can be used as the one or more fasteners. The one or more fasteners can comprise one or more of a hook and loop fastener, a button, a zipper, a snap fastener, a magnetic fastener, a safety pin, and a lace. The one or more fasteners can comprise a hook fastener on a first panel and a loop fastener on a second panel such that the first panel and second panel can be fastened together, for example, in an overlapping arrangement. The belt can have large strips of hook and loop fasteners on the closure panels and middle flaps for fastening the belt around a waist. VELCRO® (available from Velcro BVBA, Deinze, Belgium) can be used.

Different types of snaps can be attached to the different closure panels and middle flaps of the sensor belt by riveting with a punch and die set, for example, a set specific to the type of rivet snaps used. A striking action can be used on the punch using, for example, a hammer, to splay the tail. Sewing or plying with special snap pliers can also be used. Snap fasteners made out of a metal or plastic can be used.

Two or more closure panels, the mid-length section, and/or the middle flap or flaps of the sensor belt can be connected together by a line of stitching, by adhesive, or the like. Different sections, side panels, closure panels, and middle flaps can be connected together along vertical seams, for example, to connect a first side panel to a back panel or mid-length section, and to connect a second side panel to a back panel or mid-length section. Vertical seams can be used that comprise lines of stitching, lines of adhesive, lines of fasteners, lines of rivets, a combination thereof, or the like.

Nylon or other synthetic thread can be used for the stitching. Cotton thread can be used for the stitching, or any other suitable thread material. A double line of stitching can be used. Instead of, or in addition to, lines of stitching, delimiting lines can be formed using textile adhesive, fabric adhesive, hot glue gun adhesive, other adhesive, heat-bonding, heat-welding, melt-forming, other welding methods, staples, rivets, combinations thereof, and the like. Any of various commercially available fabric adhesives, stretchable or not, can be used.

Other methods and devices can be used to delineate the side panels, closure panels, middle flaps, and mid-length section of the sensor belt. Instead of, or in addition to, lines of stitching, delimiting lines can be formed using textile adhesive, fabric adhesive, hot glue gun adhesive, other adhesive, heat-bonding, heat-welding, melt-forming, other welding methods, staples, rivets, combinations thereof, and the like. Any of various commercially available fabric adhesives, stretchable or not, can be used.

One or more of the side panels, closure panels, middle flaps, and mid-length section can comprise at least one of a nylon-spandex blend material, a foamed neoprene material, an elastomeric material, elastomeric strips or patches, a combination thereof, or the like. The material can conform smoothly to a user's skin and body shape. Exemplary materials that can be used for one or more side panels, closure panels, mid-length section, and middle flaps of the sensor belt include cotton, linen, spandex, polyester, rayon, nylon, ragadon, elastone, modal, silk, satin, leather, LYCRA® (E. I. DU PONT DE NEMOURS AND COMPANY), Wilmington, Delaware, bamboo, hemp, dry-fit materials, wicking materials, breathable materials, blends of such materials, and the like materials. Organic materials can be used. The material can be comfortable.

As an example, the material can comprise cotton, polyester, nylon, spandex, LYCRA®, a foamed neoprene material, a textile material, a blend of materials, a cotton-polyester blend material, a nylon-spandex blend material, or the like. As a further example, the material can comprise at least one of a nylon-spandex blend material and a foamed neoprene material. One or more of the side panels, closure panels, mid-length section, and middle flaps can include an inner layer of a material as described above, and an outer layer. The outer layer can comprise, for example, cotton, polyester, nylon, spandex, LYCRA®, a foamed neoprene material, a textile material, a blend of materials, a cotton-polyester blend material, a nylon-spandex blend material, or the like. As a further example, the outer layer of material can comprise at least one of a nylon-spandex blend material, a foamed neoprene material, a polyester material, a cotton-polyester blend material, or the like. The outer layer of material can comprise a stretchable material.

The ultrasonic sensor and/or one or more additional sensors can be part of a control panel. The control panel can comprise, for example, a circuit board and/or an integrated circuit. The control panel can be secured to, embedded within, removably mounted on, removably mounted in, or otherwise operationally connected to the sensor belt.

The ultrasonic sensor can comprise one or more ultrasonic sensors, for example, an array of ultrasonic sensors. Exemplary ultrasonic sensors that can be implemented in the sensor belt of the present invention include those described, for example, in U.S. Patent Application Publication No. US 2010/0202253 A1 to Nakamura, U.S. Patent Application Publication No. US 2024/0015445 A1 to Schuh et al., and U.S. Patent Application Publication No. US 2019/0358387 A1 to Elbadry et al., each of which is incorporated herein by reference in its entirety.

Exemplary wetness sensors that can be implemented in the sensor belt of the present invention include those described, for example, U.S. Patent Application Publication No. US 2019/0358387 A1 to Elbadry et al., which is incorporated herein by reference in its entirety.

The sensor belt can be equipped with a BLUETOOTH (BT) radio and a cellular modem. A radiotag in the sensor belt, and a reference hub (if utilized), can have a BT radio. In addition to a BT radio, the sensor belt can have a Wi-Fi connection or serial USB connection that permits data sharing with a network host. The radiotag can transmit BT radio signals that can be received by a smart device, a physician's computer, and/or, if included, a reference hub. The radiotag can be configured to join a piconet with BT components in the sensor belt and/or with other BT devices but can also be configured so as not to form a radio link directly to a cloud server or cloud portal.

The sensor belt can include both cellular and BT radios, and a microcontroller or processor. The sensor belt can be configured to, via a cellular modem, connect via cellular radio to a cloud host with or without a virtual private gateway. The connection can be defined as a “CALL HOME”, but in the interests of saving power, the cellular modem can be used sparingly in tracking, locating, and uplinking data.

Data exchanges with the cloud from the sensor belt can generally occur over a cellular connection but can also be routed through a reference hub via a BT connection or through a WLAN radio link. Similarly, data exchanges with the cloud from the sensor belt can be routed through a smart device. In other instances, data received through a wireless connection or link from a BT piconet or other IoT sensor device can be pooled in a memory of the sensor belt or in a reference hub. Data can be shared over BT radio links, for example, and then uplinked to a cloud host via (i) a wired connection from a reference hub, (ii) via a BT connection to the sensor belt and then to a cloud host, or, (iii) via a direct cellular radio connection between the sensor belt and, for example, a cloud server. The cloud server can be representative of a class of cloud hosts termed “virtual private gateways”.

The sensor belt or a control panel for the sensor belt can comprise an accelerometer. The accelerometer can comprise an accelerometer sensor, an accelerometer processor, and an accelerometer transmitter. The accelerometer sensor can be configured to sense acceleration of motion of the sensor belt. The accelerometer processor can be configured to generate an accelerometric signal based on the sensed acceleration of motion. The accelerometer transmitter can be configured to transmit the accelerometric signal to, for example, a physician or a care team. The reminder system processor can be programmed to generate alerts pertaining to a PD therapy session, based on the data signal and the accelerometric signal. The sensor belt can be controlled to modify the PF therapy in response to the accelerometric signal.

The sensor belt can have a sensor configured to detect the progress of a therapy session, for example, the amount of time left for a fill cycle, for a dwell cycle, and/or for a drain cycle. The sensor belt can be configured to generate a data signal pertaining to the amount of time left in one or more cycles.

Other sensors that can be used with or in the sensor belt described herein include radio devices designed to detect radio traffic, such as a “ping” from a proximate radio device. Such sensors can detect received radio signal strength. Other sensors that can be used include GPS sensors having a function of fixing a location in present time, and can be combined with other data, for example, by registering a radio contact such that a sensor datum with a time stamp and a geostamp can be generated. Sensor or location data can be sent in real time without timestamp by the transmitting device or can be recorded in a memory with a timestamp for later transmission. Sensor data can be stored in a rolling sensor data log.

For any of the sensors described herein, the respective sensor can independently comprise a sensor array, a sensor matrix, a sensor pattern, a combination thereof, or the like.

According to one or more embodiments of the present invention, a sensor belt as described herein can be fastened around a patient. The back or mid-section of the sensor belt can be placed against the patient's back, the first and second panels can be wrapped around the patient's abdomen and fastened together.

A method of peritoneal dialysis treatment on the patient, is also provided. The method can comprise placing the sensor belt around the abdomen of the patient such that the first and second panels overlap with one another and rest against the abdomen of the patient. The method can include wrapping the first panel and the second panel around the patient. The method can comprise threading a catheter through a through-hole.

The catheter can have a distal end and a cap closing the distal end and the method can further comprise removing the cap prior to the filling. The method can comprise installing the cap on the distal end after the filing. The method can comprise removing the cap for a second time prior to draining. The method can comprise cleaning the distal end of the catheter prior to the filling. The catheter can include a catheter clamp thereon and the method can further comprise opening the catheter clamp prior to the filling. The method can comprise closing the catheter clamp after the filling. The method can comprise opening the catheter clamp for a second time, prior to the draining.

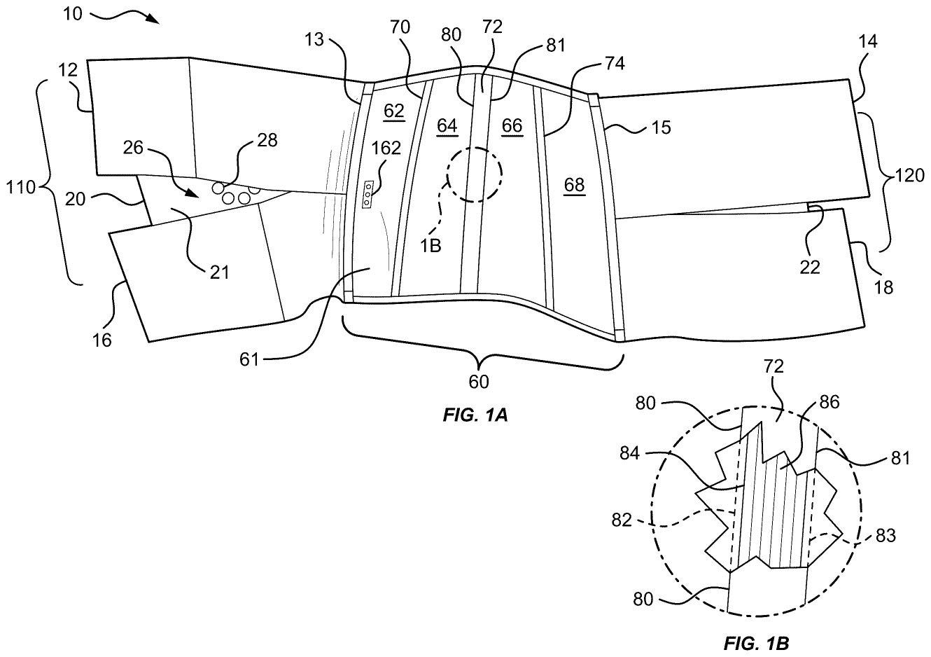

With reference now to the drawings, FIG. 1A shows an exemplary sensor belt 10 according to an embodiment of the present invention. FIG. 1A is a front view of sensor belt 10 in a laid-flat, open configuration. An enlarged sectional view is shown, in partial breakaway, in FIG. 1B that is taken from section 1B shown in FIG. 1A. Sensor belt 10 includes a mid-length section 60 in the form of a ribbed back support section. Sensor belt 10 includes a first side panel 110 and a second side panel 120. First side panel 110 comprises an upper closure panel 12 and a lower closure panel 16. A middle flap 20 is connected to the same side of mid-length section 60 where upper closure panel 12 and lower closure panel 16 are connected. Middle flap 20 forms part of a sensor panel 21 that also includes an ultrasonic sensor in the form of an array 28 of ultrasonic transceivers 28. Each of upper closure panel 12, lower closure panel 16, and middle flap 20 are secured to mid-length section 60 at a sewn seam 13 that comprises a line of stitching.

Second side panel 120 comprises an upper closure panel 14 and a lower closure panel 18. In the embodiment shown, second side panel also comprises a middle flap 22. Each of upper closure panel 14, lower closure panel 18, and middle flap 22 are secured to mid-length section 60 at a sewn seam 15 that comprises a line of stitching.

In the laid-flat configuration shown in FIG. 1A, an inner surface 61 of mid-length section 60 is shown. Mid-length section 60 is divided into back panels 62, 64, 66, and 68 that are separated from one another by ribs 70, 72, and 74. Back panel 62 includes a port 162 that can a part of or in electrical communication with a control unit (not shown) or similar logic device. Port 162 can be hardwired to ultrasonic sensor array 26 and can include or be connected to circuitry for connecting to a battery pack, circuitry for sending and receiving data, alerts, and alarms, a microprocessor, a memory, circuitry for powering a heating element, circuitry for operating a wetness sensor, circuitry for controlling a panel tensioner, a combination thereof, and the like.

Although FIG. 1A depicts ribs as 70, 72, and 74, greater details about the ribs, particularly rib 72, are shown in FIG. 1B. As can be seen in FIG. 1B, rib 72 comprises a pocket 84 in which a vertical support bar 86 is fit. Pocket 84 is defined by inner surface seams 80 and 81 and outer surface seams 82 and 83. Vertical support bars are similarly provided within respective pockets, to form ribs 70 and 74. The vertical support bars can be removable, replaceable, and interchangeable. Vertical support bars can be used that have a customized length, width, depth, stiffness, flexibility, or combination thereof. The vertical support bars can include heating elements that can be controlled by the control unit to heat mid-length section 60.

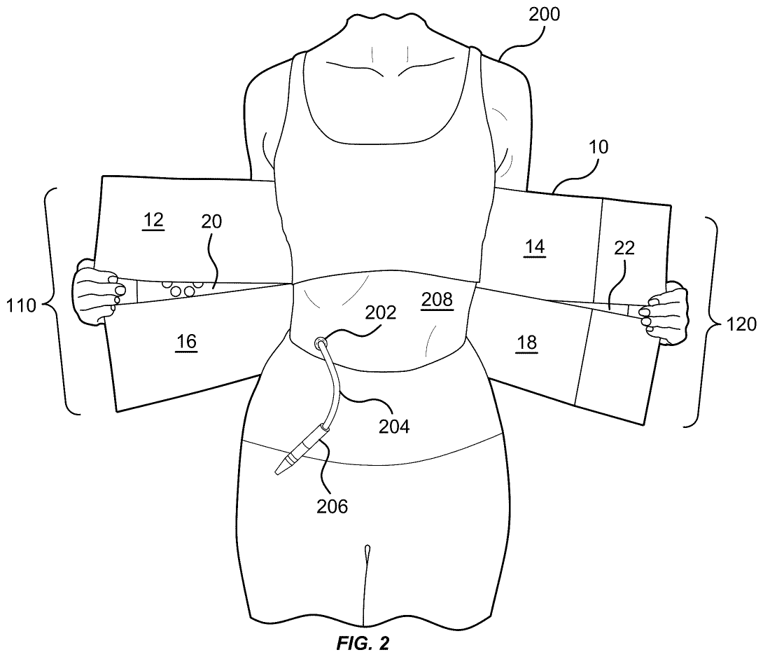

FIG. 2 depicts a first step of a method according to an embodiment of the present invention. According to the method depicted, the sensor belt shown in FIGS. 1A and 1B, is fitted around the back of a peritoneal dialysis patient 200. As can be seen, patient 200 has an abdomen 208 and a PD access 202 formed through the wall of abdomen 208. A PD catheter tube 204 is shown extending from PD access 202 and terminating at a connector 206. Although not shown, a catheter clamp can be provided along catheter tube 204.

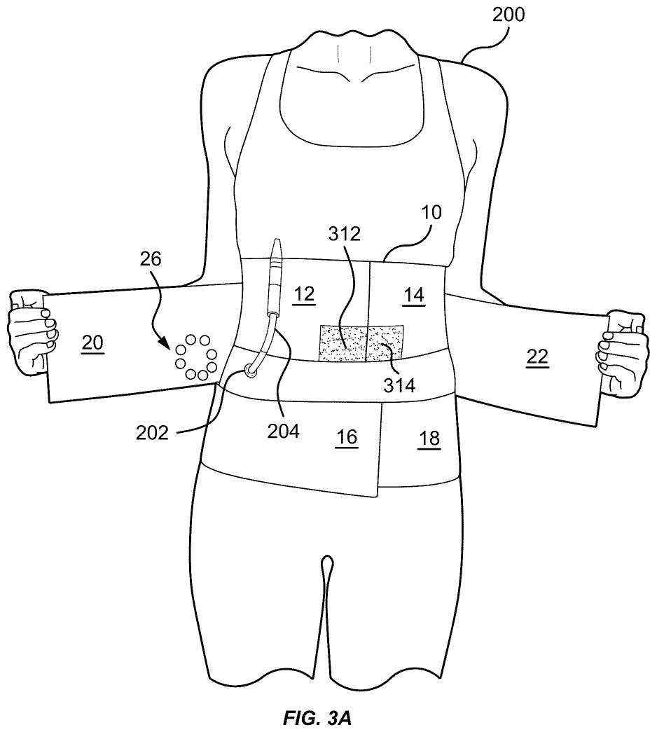

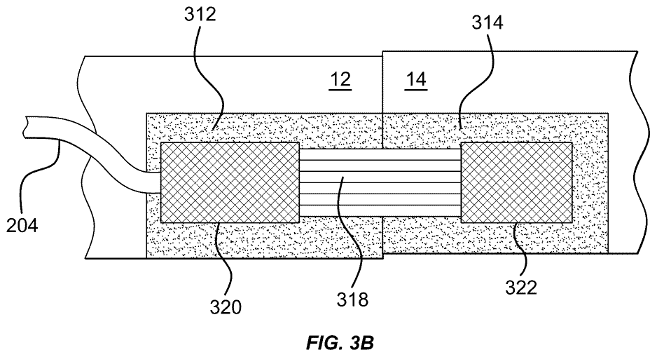

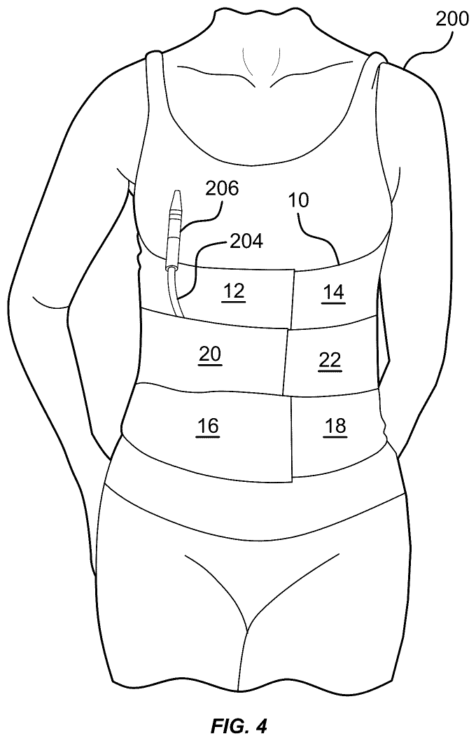

A next step of the method involves wrapping the upper closure panel 12 of first side panel 110, around the abdomen of patient 200, wrapping the upper closure panel 14 of second side panel 120, around the abdomen of patient 200, and securing upper closure panel 12 and upper closure panel 14 together, above PD access 202. Next, the method involves wrapping the lower closure panel 16 of first side panel 110, around the abdomen of patient 200, wrapping the lower closure panel 18 of second side panel 120, around the abdomen of patient 200, and securing lower closure panel 16 and lower closure panel 18 together, below PD access 202. The result of these steps is depicted in FIG. 3A. Alternatively, the lower closure panels can be secured together first, followed by securing together the upper closure panels. Either way, the result would be as shown in FIG. 3A. Neither the fastened upper closure panels nor the fastened lower closure panels covers, presses, or obstructs PD access 202 or catheter 204.

FIG. 3A also shows the outer surface of each of closure panels 12, 14, 16, and 18. As can be seen, a patch of hook and loop fasteners 312 is provided on the outer surface of upper closure panel 12 and a patch of hook and loop fasteners 314 is provided on the outer surface of upper closure panel 14. Patches 312 and 314 do not purposefully need to be configured to fasten to one another, but rather, patches 312 and 314 can be of the same material, for example, both patches 312 and 314 can comprise a patch of hook fasteners. Patches 312 and 314 can each comprise a patch of loop fasteners.

FIG. 3B depicts an intermediate step of the method, subsequent to the step shown in FIG. 3A, wherein a catheter retainer 318 has been fastened to the overlapping patches 312 and 314. In this connection, catheter retainer 318 houses the connector 206 (FIG. 2) and part of catheter 204. Catheter retainer 318 includes a first retainer sleeve 320 and a second retainer sleeve 322. Each of first retainer sleeve 320 and a second retainer sleeve 322 includes a fastener configured to fasten with patches 312 and 314, respectively. For example, the outer surface of retainer sleeve 320 can comprise a loop material and patch 312 can comprise a hook material. The outer surface of retainer sleeve 322 can comprise a loop material and patch 314 can comprise a hook material. Other fasteners can be used. Patches 312 and 314 can also be used to fasten a battery pack, a control unit, a circuit board, or the like, to sensor belt 10.

FIG. 4 is an illustration of the sensor belt shown in FIGS. 1A, 1B, 2, 3A, and 3B fastened around the back and abdomen of patient 200 with sensor panel 20, and middle flap 22 of the second side panel, being fastened to one another, with the ultrasonic sensor array aligned with the PD access of the patient. Catheter connector 206 and catheter 204 can be accessed from the top of sensor panel 20, as shown, or from below sensor panel 20 (not shown).

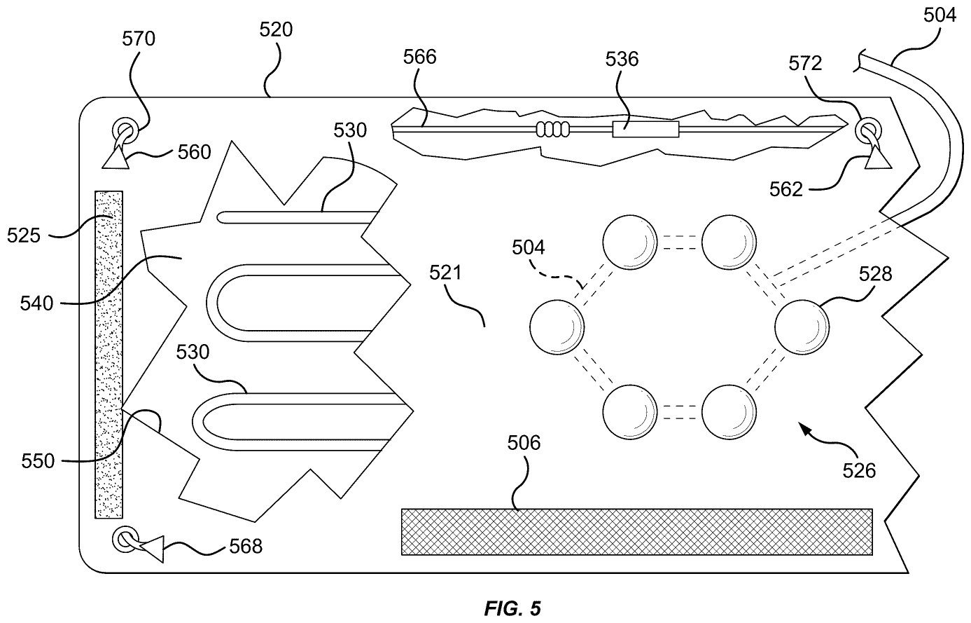

In FIG. 5, an exemplary sensor panel 520 is depicted in partial cutaway view, with an inside surface 521 shown. A hook and/or loop fastener patch 525 is provided at the distal end of inside surface 521 and is configured to be secured to or with an outermost closure flap (not shown) across the abdomen of a wearer. As can be seen from the cutaway sections, sensor panel 520 comprises an outer layer 540 and an inner layer 550. A number of elements and/or sensors can be arranged between outer layer 540 and inner layer 550, including, for example, a heating element 530 and one or more drawstrings 566 and drawstring tensioners 536. A top drawstring 566 is shown via a cutaway view and a bottom drawstring (not shown in cutaway) is also provided. The tension on top drawstring 566 can be controlled by a drawstring tensioner 536 that can comprise, for example, an electromechanical solenoid motor configured to tighten and loosen drawstring 566. A corresponding bottom arrangement (not shown), is also provided. Eyelets 570 and 572, or through-holes, and end caps 560, 562, and 568 on the drawstrings, prevent the drawstrings, including drawstring 566, from being pulled through the eyelets and provide resistance necessary to tighten or shorten the length of first panel bottom flap 520.

An array 526 of ultrasonic sensors 528 is provided and electrically connect via wiring 504 that extends to, sends signals too, and is powered by, a control board and/or integrated circuit (not shown). Wiring for tensioner 536, in this case, an electromechanical solenoid motor, along with wiring 504 and other sensor wiring and control unit wiring can be connected to the control board and/or integrated circuit. The control board and/or integrated circuit can be removable, adaptable, and/or modular. The control board and/or integrated circuit can comprise a printed circuit board.

A wetness detector 506 is arranged on a lower portion of inside surface 521, toward the bottom of sensor panel 520. Wetness detector 506 can comprise an absorbent material and can sense and send an alert signal pertaining to a degree of wetness, for example, wetness that exceeds a level of wetness attributable to normal perspiration.

The cover of each individual ultrasonic sensor 528 of ultrasonic sensor array 526, is exposed at the inside surface 521 of sensor panel 520. Each of ultrasonic sensors 528 can be in the form of an ultrasonic transceiver or pairs of ultrasonic sensors can be teamed together with one of each pair being an ultrasonic transmitter and the other of each pair being an ultrasonic receiver. For each ultrasonic transceiver or each ultrasonic transmitter/receiver pair, a logic device (not shown) is provided that generates a chirp, detects the chirp, and times the echo. Although FIG. 5 shows six ultrasonic transceivers as the ultrasonic sensor, a single ultrasonic transmitter/receiver pair can instead be used, or a single ultrasonic transceiver can instead be used.

The present invention includes the following aspects/embodiments/features in any order and/or in any combination:
1. The present invention relates to a sensor belt for peritoneal dialysis, configured to be worn by a patient during a peritoneal dialysis treatment, the patient having a back, an abdomen, and a peritoneal dialysis access formed through a region of the abdomen, the sensor belt comprising:
   a belt comprising a mid-length section having a length, a first side panel having a length, a second side panel having a length, and a middle flap, the belt having an overall length that is defined by the sum of the lengths of the mid-length section, the first side panel, and the second side panel; and
   a sensor panel comprising an ultrasonic sensor secured to the middle flap, the ultrasonic sensor being configured on or in the middle flap such that the ultrasonic sensor is aligned with and surrounds the peritoneal dialysis access when the sensor belt is worn by the patient, wherein
   the mid-length section is configured to contact the back of the patient while the patient is wearing the belt,
   the first side panel and the second side panel are provided with cooperating fasteners or fastener portions that are configured to secure the first side panel to the second side panel at the abdomen of the patient while the patient is wearing the belt,
   the first side panel comprises an upper closure panel and a lower closure panel,
   the upper closure panel is configured to wrap at least partially around the abdomen of the patient, above the peritoneal dialysis access, and fasten to the second side panel, while the patient is wearing the belt,
   the lower closure panel is configured to wrap at least partially around the abdomen of the patient, below the peritoneal dialysis access, and fasten to the second side panel, while the patient is wearing the belt,
   the upper closure panel and the lower closure panel are spaced apart from each other at the peritoneal dialysis access, such that neither the upper closure panel nor the lower closure panel covers the peritoneal dialysis access while the patient is wearing the belt, and
   the sensor panel is configured to wrap at least partially around the abdomen of the patient, cover the peritoneal dialysis access, and align the ultrasonic sensor with the region of the abdomen where the peritoneal dialysis access is located, while the patient is wearing the belt.
2. The sensor belt for peritoneal dialysis, according to any preceding or following embodiment/feature/aspect, wherein both of the first panel and second panel are made of a stretchable material.
3. The sensor belt for peritoneal dialysis, according to any preceding or following embodiment/feature/aspect, wherein the cooperating fasteners or fastener portions comprise at least one patch of hook fasteners and at least one patch of loop fasteners.
4. The sensor belt for peritoneal dialysis, according to any preceding or following embodiment/feature/aspect, wherein the cooperating fasteners or fastener portions comprise one or more of a hook and loop fastener, a button, a zipper, a snap fastener, a magnetic fastener, and a lace.
5. The sensor belt for peritoneal dialysis, according to any preceding or following embodiment/feature/aspect, wherein the ultrasonic sensor comprises an array of ultrasonic sensors arranged in a circle that is configured to surround the peritoneal dialysis access at the region of the abdomen, while the patient is wearing the belt.
6. The sensor belt for peritoneal dialysis, according to any preceding or following embodiment/feature/aspect, further comprising one or more rigid vertical support ribs configured to provided back support to the patient, wherein the mid-length section comprises one or more vertical sleeve pockets containing, respectively, the one or more rigid vertical support ribs.
7. The sensor belt for peritoneal dialysis, according to any preceding or following embodiment/feature/aspect, wherein the one or more rigid vertical support ribs are removable from the respective one or more vertical sleeve pockets.
8. The sensor belt for peritoneal dialysis, according to any preceding or following embodiment/feature/aspect, wherein
   the second side panel comprises an upper closure panel and a lower closure panel,
   the upper closure panel of the second side panel is configured to wrap at least partially around the abdomen of the patient, above the peritoneal dialysis access, and fasten to the first side panel, while the patient is wearing the belt, and
   the lower closure panel of the second side panel is configured to wrap at least partially around the abdomen of the patient, below the peritoneal dialysis access, and fasten to the first side panel, while the patient is wearing the belt.
9. The sensor belt for peritoneal dialysis, according to any preceding or following embodiment/feature/aspect, wherein neither the upper closure panel of the second side panel nor the lower closure panel of the second side panel has a length sufficient to wrap far enough around the abdomen of the patient to reach the peritoneal dialysis access.
10. The sensor belt for peritoneal dialysis, according to any preceding or following embodiment/feature/aspect, wherein, during use, the upper closure panel of the first side panel and the lower closure panel of the first side panel are vertically separated from one another by a gap, at the peritoneal dialysis access, and the sensor panel overlaps the gap and aligns the ultrasonic sensor with the peritoneal dialysis access.
11. The sensor belt for peritoneal dialysis, according to any preceding or following embodiment/feature/aspect, wherein the first side panel is connected to the mid-length section at a first vertical seam, the second side panel is connected to the mid-length section at a second vertical seam, and the middle flap is connected to the mid-length section at the first vertical seam.
12. The sensor belt for peritoneal dialysis, according to any preceding or following embodiment/feature/aspect, wherein the belt has an inner surface that is intended to contact a patient's skin, and that comprises at least one of a nylon-spandex blend material and a foamed neoprene material.
13. The sensor belt for peritoneal dialysis, according to any preceding or following embodiment/feature/aspect, wherein the ultrasonic sensor comprises an array of ultrasonic sensors arranged in a circle, and a through-hole is provided through a middle of the circle for accommodating a catheter tube.
14. In combination, the sensor belt for peritoneal dialysis, according to any preceding or following embodiment/feature/aspect, fastened around an abdomen.
15. The present invention also relates to a method for fastening the sensor belt for peritoneal dialysis, according to any preceding or following embodiment/feature/aspect, to a patient, for a peritoneal dialysis treatment of the patient, the method comprising:
   placing the mid-length section against the back of the patient;
   wrapping the first side panel and the second side panel around the abdomen of the patient;
   fastening together the first side panel and the second side panel around the abdomen of the patient; and
   wrapping the sensor panel at least partially around the abdomen of the patient and over the peritoneal dialysis access.
16. The method of any preceding or following embodiment/feature/aspect, further comprising:
   sending ultrasonic waves from the ultrasonic sensor toward the region of the abdomen where the peritoneal dialysis access is formed; and
   receiving, with the ultrasonic sensor, ultrasonic signals reflected from musculature within the abdomen of the patient.
17. The method of any preceding or following embodiment/feature/aspect, further comprising carrying out a peritoneal dialysis treatment on the patient, through the peritoneal dialysis access, while receiving, with the ultrasonic sensor, ultrasonic signals reflected from the musculature within the abdomen of the patient.
18. The method of any preceding or following embodiment/feature/aspect, further comprising modifying the peritoneal dialysis treatment, based on the ultrasonic signals received.
19. The method of any preceding or following embodiment/feature/aspect, wherein the sensor belt further comprises a heater, and the method further comprises heating the belt during the peritoneal dialysis treatment.
20. The present invention also relates to a method for carrying out a peritoneal dialysis treatment on a patient, the method comprising:
   fastening a sensor belt around the abdomen of the patient, the sensor belt comprising an ultrasonic sensor;
   aligning the ultrasonic sensor of the sensor belt with a peritoneal dialysis access formed in the abdomen;
   carrying out a peritoneal dialysis treatment on the patient by transferring a peritoneal dialysis solution through the peritoneal dialysis access;
   sending and receiving ultrasonic signals from and to the ultrasonic sensor, the ultrasonic signals received pertaining to the musculature of the abdomen of the patient; and modifying the peritoneal dialysis treatment based on the ultrasonic signals received.

The entire contents of all references cited in this disclosure are incorporated herein in their entireties, by reference. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether such a range is separately disclosed. Where a range of numerical values is recited herein, unless otherwise stated, the range is intended to include the endpoints thereof, and all integers and fractions within the range. It is not intended that the scope of the invention be limited to the specific values recited when defining a range.

All patents, patent applications, and publications mentioned herein are incorporated herein in their entireties, by reference, unless indicated otherwise.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A sensor belt for peritoneal dialysis, configured to be worn by a patient during a peritoneal dialysis treatment, the patient having a back, an abdomen, and a peritoneal dialysis access formed through a region of the abdomen, the sensor belt comprising:
a belt comprising a mid-length section having a length, a first side panel having a length, a second side panel having a length, and a middle flap, the belt having an overall length that is defined by the sum of the lengths of the mid-length section, the first side panel, and the second side panel; and
a sensor panel comprising an ultrasonic sensor secured to the middle flap, the ultrasonic sensor being configured on or in the middle flap such that the ultrasonic sensor is aligned with and surrounds the peritoneal dialysis access when the sensor belt is worn by the patient, wherein
the mid-length section is configured to contact the back of the patient while the patient is wearing the belt,
the first side panel and the second side panel are provided with cooperating fasteners or fastener portions that are configured to secure the first side panel to the second side panel at the abdomen of the patient while the patient is wearing the belt,
the first side panel comprises an upper closure panel and a lower closure panel,
the upper closure panel is configured to wrap at least partially around the abdomen of the patient, above the peritoneal dialysis access, and fasten to the second side panel, while the patient is wearing the belt,
the lower closure panel is configured to wrap at least partially around the abdomen of the patient, below the peritoneal dialysis access, and fasten to the second side panel, while the patient is wearing the belt,
the upper closure panel and the lower closure panel are spaced apart from each other at the peritoneal dialysis access, such that neither the upper closure panel nor the lower closure panel covers the peritoneal dialysis access while the patient is wearing the belt, and
the sensor panel is configured to wrap at least partially around the abdomen of the patient, cover the peritoneal dialysis access, and align the ultrasonic sensor with the region of the abdomen where the peritoneal dialysis access is located, while the patient is wearing the belt.

2. The sensor belt for peritoneal dialysis of claim 1, wherein both of the first panel and second panel are made of a stretchable material.

3. The sensor belt for peritoneal dialysis of claim 1, wherein the cooperating fasteners or fastener portions comprise at least one patch of hook fasteners and at least one patch of loop fasteners.

4. The sensor belt for peritoneal dialysis of claim 1, wherein the cooperating fasteners or fastener portions comprise one or more of a hook and loop fastener, a button, a zipper, a snap fastener, a magnetic fastener, and a lace.

5. The sensor belt for peritoneal dialysis of claim 1, wherein the ultrasonic sensor comprises an array of ultrasonic sensors arranged in a circle that is configured to surround the peritoneal dialysis access at the region of the abdomen, while the patient is wearing the belt.

6. The sensor belt for peritoneal dialysis of claim 1, further comprising one or more rigid vertical support ribs configured to provided back support to the patient, wherein the mid-length section comprises one or more vertical sleeve pockets containing, respectively, the one or more rigid vertical support ribs.

7. The sensor belt for peritoneal dialysis of claim 6, wherein the one or more rigid vertical support ribs are removable from the respective one or more vertical sleeve pockets.

8. The sensor belt for peritoneal dialysis of claim 1, wherein
the second side panel comprises an upper closure panel and a lower closure panel,
the upper closure panel of the second side panel is configured to wrap at least partially around the abdomen of the patient, above the peritoneal dialysis access, and fasten to the first side panel, while the patient is wearing the belt, and
the lower closure panel of the second side panel is configured to wrap at least partially around the abdomen of the patient, below the peritoneal dialysis access, and fasten to the first side panel, while the patient is wearing the belt.

9. The sensor belt for peritoneal dialysis of claim 8, wherein neither the upper closure panel of the second side panel nor the lower closure panel of the second side panel has a length sufficient to wrap far enough around the abdomen of the patient to reach the peritoneal dialysis access.

10. The sensor belt for peritoneal dialysis of claim 1, wherein, during use, the upper closure panel of the first side panel and the lower closure panel of the first side panel are vertically separated from one another by a gap, at the peritoneal dialysis access, and the sensor panel overlaps the gap and aligns the ultrasonic sensor with the peritoneal dialysis access.

11. The sensor belt for peritoneal dialysis of claim 1, wherein the first side panel is connected to the mid-length section at a first vertical seam, the second side panel is connected to the mid-length section at a second vertical seam, and the middle flap is connected to the mid-length section at the first vertical seam.

12. The sensor belt for peritoneal dialysis of claim 1, wherein the belt has an inner surface that is intended to contact a patient's skin, and that comprises at least one of a nylon-spandex blend material and a foamed neoprene material.

13. The sensor belt for peritoneal dialysis of claim 1, wherein the ultrasonic sensor comprises an array of ultrasonic sensors arranged in a circle, and a through-hole is provided through a middle of the circle for accommodating a catheter tube.

14. In combination, the sensor belt for peritoneal dialysis of claim 1 fastened around an abdomen.

15. A method for fastening the sensor belt for peritoneal dialysis, of claim 1, to a patient, for a peritoneal dialysis treatment of the patient, the method comprising:
placing the mid-length section against the back of the patient;
wrapping the first side panel and the second side panel around the abdomen of the patient;
fastening together the first side panel and the second side panel around the abdomen of the patient; and
wrapping the sensor panel at least partially around the abdomen of the patient and over the peritoneal dialysis access.

16. The method of claim 15, further comprising:
sending ultrasonic waves from the ultrasonic sensor toward the region of the abdomen where the peritoneal dialysis access is formed; and
receiving, with the ultrasonic sensor, ultrasonic signals reflected from musculature within the abdomen of the patient.

17. The method of claim 16, further comprising carrying out a peritoneal dialysis treatment on the patient, through the peritoneal dialysis access, while receiving, with the ultrasonic sensor, ultrasonic signals reflected from the musculature within the abdomen of the patient.

18. The method of claim 17, further comprising modifying the peritoneal dialysis treatment, based on the ultrasonic signals received.

19. The method of claim 17, wherein the sensor belt further comprises a heater, and the method further comprises heating the belt during the peritoneal dialysis treatment.

20. A method for carrying out a peritoneal dialysis treatment on a patient, the method comprising:
fastening a sensor belt around the abdomen of the patient, the sensor belt comprising an ultrasonic sensor;
aligning the ultrasonic sensor of the sensor belt with a peritoneal dialysis access formed in the abdomen;
carrying out a peritoneal dialysis treatment on the patient by transferring a peritoneal dialysis solution through the peritoneal dialysis access;
sending and receiving ultrasonic signals from and to the ultrasonic sensor, the ultrasonic signals received pertaining to the musculature of the abdomen of the patient; and
modifying the peritoneal dialysis treatment based on the ultrasonic signals received.
